(19) United States
(12) Patent Application Publication
(10) Pub. No.: US 2026/0114717 A1
(43) Pub. Date: Apr. 30, 2026

(54) WORKING CHANNEL TOOL INTRODUCER

(71) Applicant: Auris Health, Inc., Santa Clara, CA (US)

(72) Inventors: Boney Charlie MEJIA, Mountain View, CA (US); Grace Magdalena RESTLE, San Francisco, CA (US); Robert Mason GLIDDEN, IV, Emerald Hills, CA (US)

(73) Assignee: Auris Health, Inc., Santa Clara, CA (US)

(21) Appl. No.: 19/365,933

(22) Filed: Oct. 22, 2025

Related U.S. Application Data
(60) Provisional application No. 63/711,516, filed on Oct. 24, 2024.

(51) Int. Cl. A61B 1/00

(52) U.S. Cl. CPC A61B 1/00131; A61B 1/00128

(57) ABSTRACT

A tool introducer assembly includes a valve having a valve head that provides a seal, and an introducer that includes a column that defines a channel that extends therethrough, and a securing mechanism configured to attach the introducer to the valve. The valve and the introducer are slidably coupled with a range of motion limited by the securing mechanism, and, when the valve and the introducer are coupled, the column of the introducer is aligned with an opening defined by the seal.

### BACKGROUND

Certain robotic medical procedures can involve the use of shaft-type instruments, such as endoscopes, which may be inserted into a patient through an orifice (e.g., a natural orifice) and advanced to a target site. Such medical instruments can include a working channel and a medical tool may be introduced through the working channel to the target site.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

Embodiments disclosed herein include a tool introducer assembly that includes a valve having a valve head that provides a seal, and an introducer that includes a column that defines a channel that extends therethrough, and a securing mechanism configured to attach the introducer to the valve, wherein the valve and the introducer are slidably coupled with a range of motion limited by the securing mechanism, and wherein, when the valve and the introducer are coupled, the column of the introducer is aligned with an opening defined by the seal.

Embodiments disclosed herein further include a device for introducing a flexible elongate tool into a valve, the device including a column molded on and extending from a surface and defining a channel that extends through the column, a set of walls extending from the surface parallel to and at least partially surrounding the column, the set of walls terminating at and forming a crescent at a side opposite the surface in relation to the column, the crescent providing an opposing surface that is parallel to the surface, and at least one latch formed on the crescent and facing radially inward and toward the column.

Embodiments disclosed herein further include a method of introducing a working channel tool into a valve, the method including assembling an introducer with the valve to obtain an introducer assembly, the introducer including a column that defines a channel that extends therethrough, and at least one latch that secures the introducer to the valve. The method further including sliding the introducer toward the valve and thereby opening a seal on the valve as the column penetrates an opening defined in the seal, sliding the introducer away from the valve and thereby closing the seal as the column withdraws from the opening, and preventing the introducer from decoupling from the valve with the at least one latch.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example of a robotic medical system including a shaft-type instrument coupled to a robotic end effector, in accordance with some implementations.

FIG. 2A-2B illustrate medical system components that may be implemented in the robotic medical system of FIG. 1, in accordance with some implementations.

FIG. 3 illustrates a shaft-type instrument disposed in a subject, in accordance with some implementations.

FIG. 4 illustrates a view of an example working channel tool assembly, in accordance with some implementations.

FIG. 5A-5B illustrate an unassembled configuration and an assembled configuration, respectively, of an example instrument handle, in accordance with some implementations.

FIG. 6A-6D illustrate various views of an example introducer, in accordance with some implementations.

FIG. 7 illustrates a cross-sectional view of the example introducer of FIGS. 6A-6D, in accordance with some implementations.

FIG. 8A-8C illustrate various configurations of an example introducer assembly including a valve and an introducer, in accordance with some implementations.

FIG. 9 illustrates a flow diagram for an example process for assembling and operating the example introducer assembly of FIG. 8A-8C, in accordance with some implementations.

FIG. 10A-10H illustrate example introducers having various latching designs, in accordance with some implementations.

FIG. 11A-11D illustrate various views of the latching design shown in FIG. 10H, in accordance with some implementations.

FIG. 12A-12D illustrate example introducers having various press-fit designs, in accordance with some implementations.

FIG. 13A-13C illustrate example introducers having various O-ring-based designs, in accordance with some implementations.

FIG. 14A-14B illustrate example introducers having various magnetic designs, in accordance with some implementations.

FIG. 15A-15B illustrate example hubs to which an introducer can be releasably coupled, in accordance with some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts, features associated with separate figures that are identified by common reference numbers are not related and/or similar with respect to at least certain aspects.

The present disclosure provides systems, devices, and methods relating to introducing a medical tool into a working channel of a robotically (and manually) articulable shaft-like medical instrument. The shaft-like medical instrument can be an endoscope navigated to a site within a subject and the introduced medical tool may reach the site through the working channel. In some examples, the medical tool can be a laser tool for fragmenting a kidney stone, a basket tool for grabbing and removing the fragments, or the like. The medical tool (also referred as a “working channel tool”) may be introduced into a proximal opening of the working channel and slide within the working channel toward the site when inserted and away from the site (toward the proximal opening) when retracted. In some examples, a valve may be positioned at the proximal opening such that the medical tool may be introduced into or removed from the working channel via the valve. The valve may be a hemostatic valve (also generally referred as a hemostasis valve) configured to prevent loss of fluids (e.g., blood, saline solution, irrigation/aspiration fluid, or the like) with its seal while allowing the medical tool to pass through the seal. An introducer may facilitate sanitized introduction of the medical tool into the hemostatic valve by opening the seal during the introduction. The introducer as a separate tool may be lost or become contaminated when dropped. An improved introducer disclosed herein may be provided as a permanent or semi-permanent assembly with the hemostatic valve, thereby addressing the above issues.

### Medical Procedures

Although certain aspects of the present disclosure are described in detail herein in the context of renal, urological, and/or nephrological procedures, such as kidney stone removal/treatment procedures, it should be understood that such context is provided for convenience and clarity, and the working channel tool assembly concepts and designs disclosed herein are applicable to any suitable robotic medical instruments used in various medical procedures, such as robotic bronchoscopy. However, as mentioned, description of the renal/urinary anatomy and associated medical issues and procedures is presented below to aid in the description of the inventive concepts disclosed herein.

In certain medical procedures, such as ureteroscopy procedures, elongate medical instruments that access the treatment site through an access sheath may be utilized to remove debris, such as kidney stones and stone fragments or other refuse or contaminant(s), from the treatment site. Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis. ” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shock wave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or through aspiration.

In some procedures, surgeons may insert an endoscope (e.g., ureteroscope) into the urinary tract through the urethra to remove urinary stones from the bladder and ureter. Typically, a ureteroscope includes a camera proximate its distal end configured to enable visualization of the urinary tract. The ureteroscope can also include, or allow for placement in a working channel of the ureteroscope, a lithotripsy device configured to capture or break apart urinary stones. During a ureteroscopy procedure, one physician/technician may control the position of the ureteroscope, while another physician/technician may control the lithotripsy device(s).

In some procedures, such as procedures for removing relatively large stones/fragments, physicians may use a percutaneous nephrolithotomy (“PCNL”) technique that involves inserting a nephroscope through the skin (i.e., percutaneously) and intervening tissue to provide access to the treatment site for breaking-up and/or removing the stone(s). A percutaneous-access device (e.g., nephroscope, sheath, sheath assembly, and/or catheter) used to provide an access channel to the target anatomical site (and/or a direct-entry endoscope) may include one or more fluid channels for providing irrigation fluid flow to the target site and/or aspirating fluid from the target site (e.g., through passive outflow and/or active suction).

For ureteroscopic procedures, a physician may implement a procedure to break a relatively large kidney stone into relatively smaller fragments to facilitate extraction thereof. For example, certain instruments may be utilized to break the stone into smaller fragments, such as by lasing, or through other application of cleaving force to the kidney stone. According to some procedures, a basketing device/system may be used to capture the relatively smaller stone fragment(s) and extract them from the treatment site out of the patient. Generally, when a stone is captured, the surgeon may wish to quickly extract the stone through the ureteral access sheath prior to opening the basket to deposit/drop the stone into a specimen collection structure or area, after which the basket may be closed and reinserted (e.g., within a working channel of an endoscope/ureteroscope) through the access sheath for the purpose of extracting remaining stones or stone fragments, should there be any.

Robotic-assisted ureteroscopic procedures can be implemented in connection with various medical procedures, such as kidney stone removal procedures, wherein robotic tools can enable a physician/urologist to perform endoscopic target access as well as percutaneous access/treatment. Advantageously, aspects of the present disclosure relate to systems, devices, and methods for robotically controlling insertion, retraction, and operation of various medical tools that can be navigated through a working channel of an endoscope.

### Medical System

Various aspects of the present disclosure described herein may be integrated into a robotically enabled/assisted medical system, including a surgical robotic system (or “robotic system”), capable of performing a variety of medical procedures, including both minimally invasive (e.g., laparoscopy) and non-invasive (e.g., endoscopy) procedures. Among endoscopy procedures, the robotically enabled medical system may be capable of performing bronchoscopy, ureteroscopy, gastroscopy, etc.

In addition to performing the breadth of procedures, the robotically enabled medical system may provide additional benefits, such as enhanced imaging and guidance to assist the medical professional. Additionally, the robotically enabled medical system may provide the medical professional with the ability to perform the procedure from an ergonomic position without the need for awkward arm motions and positions. Still further, the robotically enabled medical system may provide the medical professional with the ability to perform the procedure with improved ease of use such that one or more of the instruments of the robotically enabled medical system may be controlled by a single operator.

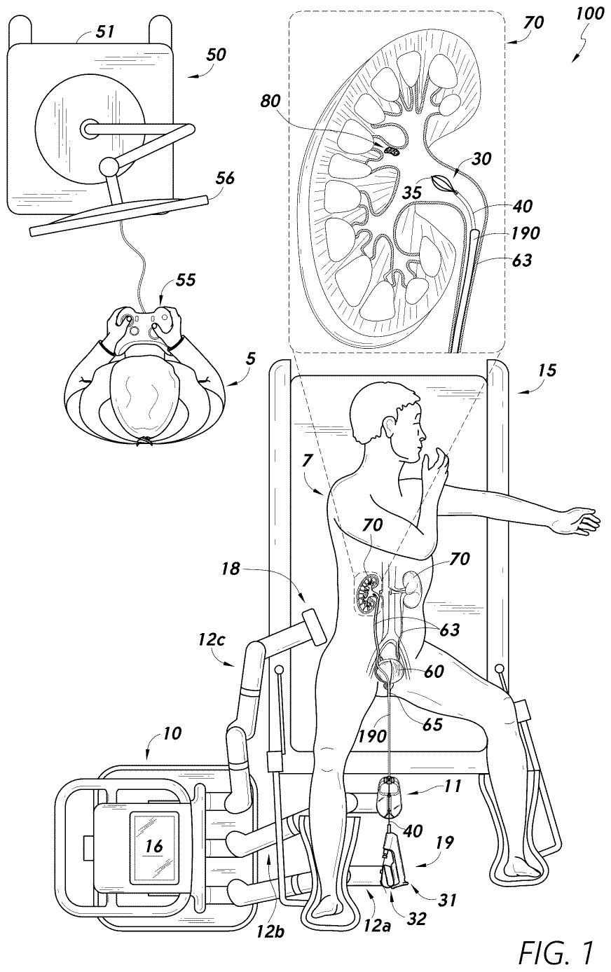

FIG. 1 illustrates an example medical system 100 for performing various medical procedures in accordance with aspects of the present disclosure. The medical system 100 may be used for, for example, endoscopic (e.g., ureteroscopic) procedures. The principles disclosed herein may be implemented in any type of endoscopic (e.g., bronchial, gastrointestinal, etc.) and/or percutaneous procedure.

The medical system 100 includes a robotic system 10 (e.g., a mobile robotic cart as shown, a table-based system with integrated robotic arms, etc.) that is configured to engage with and/or control a medical instrument 19 (e.g., ureteroscope) including a proximal instrument base 31 and a shaft 40 coupled to the instrument base 31 at a proximal portion thereof to perform a direct-entry procedure on a subject 7. The term “subject” is used herein to refer to live patient and human anatomy as well as any subjects to which the present disclosure may be applicable. For example, the “subject” may refer to subjects including physical anatomic models (e.g., anatomical education model, anatomical model, medical education anatomy model, etc.) used in dry runs, models in computer simulations, or the like that cover non-live patients or test subjects. The term “direct-entry” is used herein according to its broad and ordinary meaning and may refer to any entry of instrumentation through a natural or artificial opening in a patient's body. For example, with reference to FIG. 1, the direct entry of the scope/shaft 40 into a urinary tract 63 of the subject 7 may be made via the urethra 65.

It should be understood that the medical instrument 19 may be any type of shaft-based medical instrument, including an endoscope (such as a ureteroscope), a catheter (such as a steerable or non-steerable catheter), a nephroscope, a laparoscope, or another type of medical instrument. The medical instruments disclosed herein may be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body.

The medical instrument 19 has a flexible elongated body that has mechanical couplings which enable the elongated body to flex, bend, deflect or articulate to some angle, in response to an actuator (e.g., containing a motor) being energized in accordance with a command (also referred to as an input, which refers to (e.g., defines) a desired direction or a desired articulation angle, for example). An example of such a medical instrument 19 is a flexible endoscope (or scope) that may be any type of elongated medical instrument having image generating, viewing, and/or capturing functionality and configured to be introduced into any type of organ, cavity, lumen, chamber, or space of a patient's body. The scope 40 may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon and/or rectum), an arthroscope (e.g., for accessing a joint), a cystoscope (e.g., for accessing the bladder), a borescope, and so on. The elongated body may comprise a flexible tube or shaft and may be dimensioned to be passed within an outer sheath, catheter, introducer, or other lumen-type device, or it may be used without such devices.

In an example use case, if the subject 7 has a kidney stone (or stone fragment) 80 located in a kidney 70, the operator 5 may perform a procedure to remove the stone 80 through the urinary tract 63 using a basket 35. In some examples, the operator 5 can interact with a control system 50 and/or the robotic system 10 to cause/control the robotic system 10 to advance and navigate the medical instrument 19 through the calyx network of the kidney 70 to where the stone 80 is located. The control system 50 can provide information via one or more display(s) 56 associated with the medical instrument 19, such as real-time endoscopic images captured therewith, and/or other instruments of the medical system 100, to assist the operator 5 in navigating/controlling such instrumentation.

The medical system 100 shown as an example in the figures further includes a table 15, and an electromagnetic (EM) field generator 18. Table 15 is configured to support the subject 7 for example, as shown. The EM field generator 18 may be held by one of the robotic arms 12c of the robotic system 10, may be a stand-alone device, or may be integrated into the table 15. In some versions, the table 15 has actuators that can change, for example, the height and orientation of the table 15. IN such applications, the control system 50 may communicate with the table 15 to position the table 15 in a particular orientation or otherwise control the orientation of table 15.

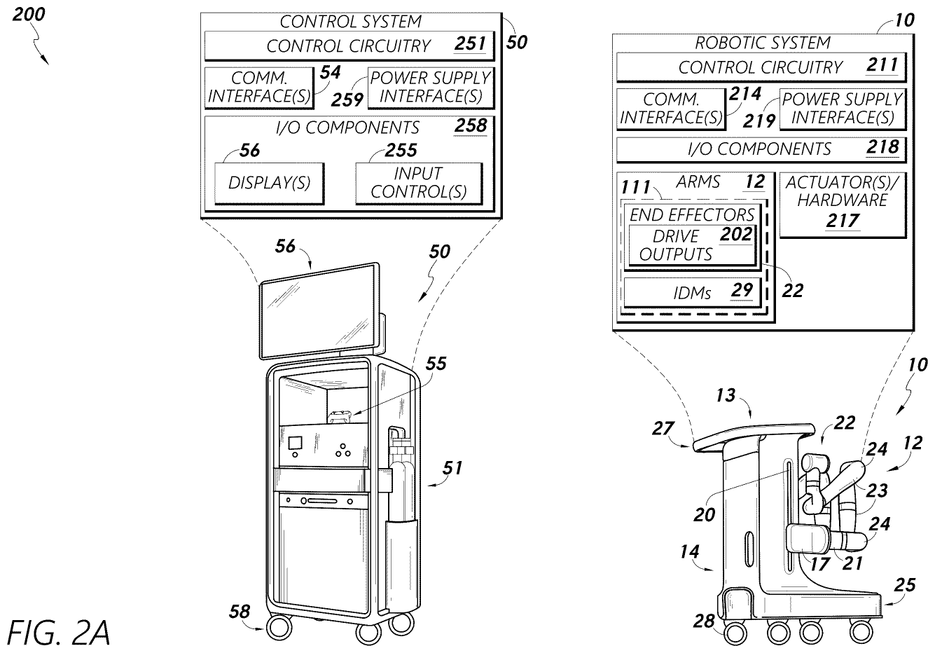

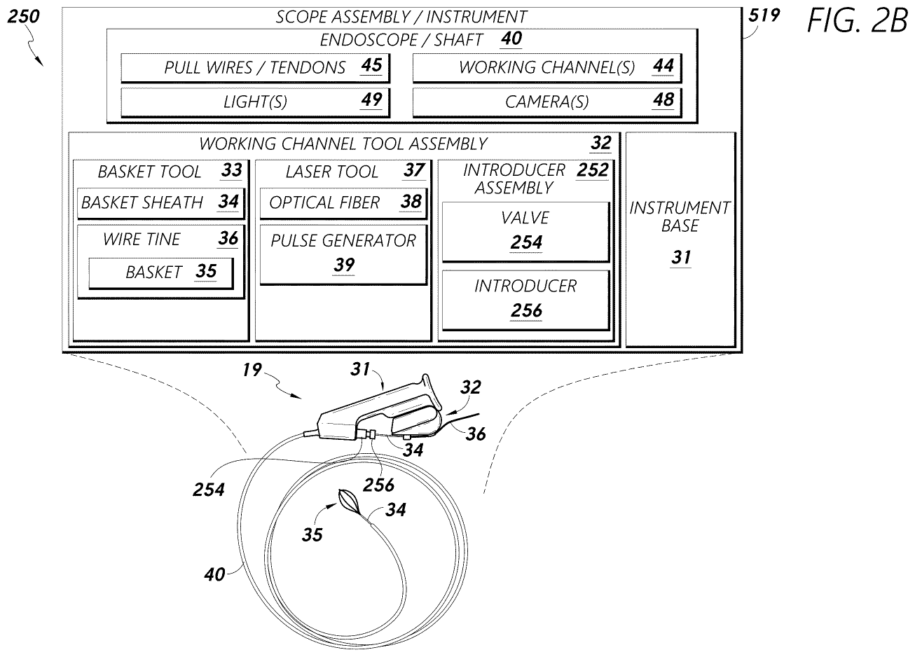

As shown in FIG. 2A, the medical system 100 includes the control system 50 configured to interface with the robotic system 10, provide information regarding the procedure, and/or perform a variety of other operations. The control system 50 of the present example includes various input/output (I/O) components 258 configured to assist the operator 5 or others in performing a medical procedure. For example, the I/O components 258 may be configured to allow for user input to control/navigate the medical instrument 19 and any components thereof within the subject 7. I/O components 258 of the present example include a controller 55 that is configured to receive user input from the operator 5 and a display 56 configured to present certain information to assist the operator. The controller 55 may take any suitable form including, but not limited to, one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, etc.

The control system 50 of the present example includes a communication interface 54 that is operable to provide a communicative interface between the control system 50 and the robotic system 10, the medical instrument 19, and/or other components. Communications via the communication interface 54 may include data, commands, electrical power, and/or other forms of communication. The communication interface 54 may also be configured to provide communication via wired, wireless, and/or other communication modalities. The control system 50 also includes a power supply interface 259, which may receive power to the drive control system 50 via wire, battery, and/or any other suitable kind of power source. A control circuitry 251 of the control system 50 may provide signal processing and execute control algorithms to achieve the functionality of the medical system 100 as described herein.

The control system 50 may also communicate with the robotic system 10 to receive pose data therefrom relating to the pose (e.g., position and/or orientation) of the distal end of the medical instrument flexible elongated body. Such pose data may be derived using one or more EM sensors that may be mounted to the flexible elongated body of the medical instrument 19, and that interact with an EM field generated by the EM field generator 18. The control system 50 may communicate with the EM field generator 18 to control the generation of the EM field in an area around the subject 7. Other ways of detecting the pose (e.g., 3D position and/or orientation) of the distal end of the medical instrument 19 are possible, such as using an optical camera/imaging-based system.

As noted above and as shown in FIG. 1 and FIG. 2A-2B, the robotic system 10 includes robotic arms 12 that are configured to engage with and/or control the medical instrument 19 to perform one or more aspects of a procedure. It should be understood that a given robotic arm 12 may be coupled to instruments that are different than those (e.g., introducer for guiding the medical instrument 19 to an access point, such as the urethra 65) shown in FIG. 1; and in some scenarios, one or more of the robotic arms 12 may not be utilized or coupled to a medical instrument. As shown in FIG. 2A, each robotic arm 12 includes multiple linking arm segments 23 coupled to joints 24, which enable the attached medical instrument to have multiple degrees of movement/freedom. In the example of FIG. 1, the robotic system 10 is positioned proximate to the patient's legs and the robotic arms 12 are actuated to engage with and position the medical instrument 19 for access into an access opening of the subject 7. When the robotic system 10 is properly positioned, the medical instrument 19 may be inserted into the subject 7 robotically using the robotic arms 12, manually by the operator 5, or a combination thereof.

The robotic system 10 may be coupled to any component of the medical system 100, such as the control system 50, the table 15, the EM field generator 18, the medical instrument 19, and/or any type of percutaneous-access instrument (e.g., needle, catheter, nephroscope, etc.). As noted above, the robotic system 10 may be communicatively coupled with the control system 50 via communication interfaces 214, 54. Robotic system 10 also includes a power supply interface 219, which may receive power to drive the robotic system 10 via wire, battery, and/or any other suitable kinds of power sources. In addition, the robotic system 10 example includes various input/output (I/O) components 218 configured to assist the operator 5 or others in performing a medical procedure. Such I/O components 218 may include any of the various kinds of I/O components 258 described later with greater detail in the context of the control system 50. In addition, or in the alternative, I/O components 218 of robotic system 10 may take any suitable form (or may be omitted altogether).

The robotic system 10 of the present example generally includes a robotic system base 25, a column 14, and a console 13 at the top of the column 14. The robotic system base 25 balances the weight of the column 14, an arm support 17, and the robotic arms 12 over the floor. Accordingly, the robotic system base 25 may house certain relatively heavier components, such as electronics, motors, a power supply, as well as components that selectively enable movement or non-movement of the robotic system 10. For example, the robotic system base 25 can include wheel-shaped casters 28 that allow for the robotic system 10 to easily move around the operating room prior to a procedure. After reaching the appropriate position, the casters 28 may be immobilized using wheel locks to hold the robotic system 10 in place during the procedure.

Positioned at the upper end of the column 14, the console 13 can provide both a user interface (e.g., I/O components 218) for receiving user input and a display screen 16 (or a dual-purpose device such as, for example, a touchscreen) to provide the physician/user with both pre-operative and intra-operative data. As shown, the console 13 can also include a handle 27 to assist with maneuvering and stabilizing the robotic system 10. The column 14 may include one or more arm supports 17 for supporting the deployment of the one or more robotic arms 12 (where three robotic arms 12a, 12b, 12c are shown in FIG. 1). The arm support 17 may include individually-configurable arm mounts that rotate along a perpendicular axis to adjust the base of the robotic arms 12 for desired positioning relative to the subject 7.

The arm support 17 may be configured to vertically translate along the column 14. In some examples, the arm support 17 can be connected to the column 14 through slot 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, subject sizes, and physician preferences. Similarly, the individually-configurable arm mounts on the arm support 17 can allow the robotic arm base 21 of each robotic arm 12 to be angled in a variety of configurations.

The robotic arms 12 may generally include robotic arm bases 21 and end effectors 22, separated by a series of linking arm segments 23 that are connected by a series of joints 24, each joint 24 comprising one or more independent actuators 217. Each actuator may comprise an independently-controllable motor. Each joint 24 may be independently-controllable and can provide or represent an independent degree of freedom available to the corresponding robotic arm 12. In some examples, each of the robotic arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the subject 7 to create greater access, while avoiding arm collisions.

The term “end effector” is used herein according to its broad and ordinary meaning and may refer to any type of robotic manipulator device, component, and/or assembly. Where an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly, wherein such assembly may or may not also include a medical instrument (or instrument handle/base) physically coupled to the adapter and/or end effector. The terms “robotic manipulator” and “robotic manipulator assembly” are used according to their broad and ordinary meanings, and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, sterile adapter, and/or other component(s).

The end effector 22 of each robotic arm 12 may include an instrument device manipulator (IDM) 29, which may be attached using a mechanism changer interface (MCI). The MCI may provide power and control interfaces (e.g., connectors to transfer pneumatic pressure, electrical power, electrical signals, and/or optical signals from the robotic arm 12) to the IDM 29. In some examples, the IDM 29 may be removed and replaced with a different type of IDM 29, depending on the type of the medical instrument 19 that is to be attached to the arm. Each type of IDM 29 may serve to manipulate a respective type of the medical instrument 19. In the case where the medical instrument 19 is a scope, the IDM 29 may use any one or combination of techniques including, for example, direct drives, harmonic drives, geared drives, belts and pulleys, magnetic drives, and the like, to drive the flexible elongated body of the scope so that the distal end is positioned to some desired angle or bent in some desired direction. A second type of IDM 29 may manipulate a basketing system or a steerable catheter by driving the flexible elongated body of the catheter or basketing system so that the distal end is positioned at some angle. Another type of IDM 29 may be configured to hold the EM field generator 18. Many variations are possible.

The medical system 100 may include certain control circuitry configured to perform certain of the functionality described herein, including the control circuitry 211 of the robotic system 10 and the control circuitry 251 of the control system 50. That is, the control circuitry 211, 251 of the medical system 100 may be part of the robotic system 10, the control system 50, or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry 211, 251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

In some examples, for example, the operator 5 may provide input to the control system 50 and/or robotic system 10, and in response to such input, control signals may be sent to the robotic system 10 to manipulate the medical instrument 19. The control system 50 may include one or more display devices (e.g., the display 56) to provide various information regarding a procedure. For example, the display 56 may provide information regarding the medical instrument 19. In the case of a scope, the control system 50 may receive real-time images that are captured by the scope representing internal anatomy of the subject 7 and display the real-time images via the display 56.

FIG. 2B illustrates a scope assembly 519 as the medical instrument 19 shown in FIG. 1, in accordance with some implementations. The endoscope (i.e., “scope” or “shaft”) can include an elongate shaft including one or more lights 49 and one or more cameras or other imaging devices 48, which may be integrated as a part of the endoscope or provided as a separate camera assembly. The scope 40 can further include one or more working channels 44, which may run a length of the scope 40. In some examples, such channel(s) may be utilized to provide access for a working channel tool (e.g., a basket tool 33, a laser tool 37, forceps, the camera assembly, etc.) for introduction through the scope 40 to the treatment site.

The scope assembly 519 may comprise an instrument base 31 for the scope 40, wherein the scope 40 is coupled to the instrument base 31 at a proximal end of the scope 40. The instrument base 31 can be shaped and configured as a handle to provide scope control either manually or robotically. In some examples, a working channel tool assembly 32 may releasably attach to the instrument base 31 and be configured to provide control of the working channel tool, either manually or robotically, in relation to the working channel 44 of the scope 40. When attached, the instrument base 31 and the working channel tool assembly 32 can operate as a single instrument unit (referred herein as an “instrument handle”) to be mounted on and operated by an end effector 22 of a single robotic arm 12a (FIG. 1). The releasable attachment supports modular design of the instrument handle that, advantageously, help isolate working channel tool control from the scope control.

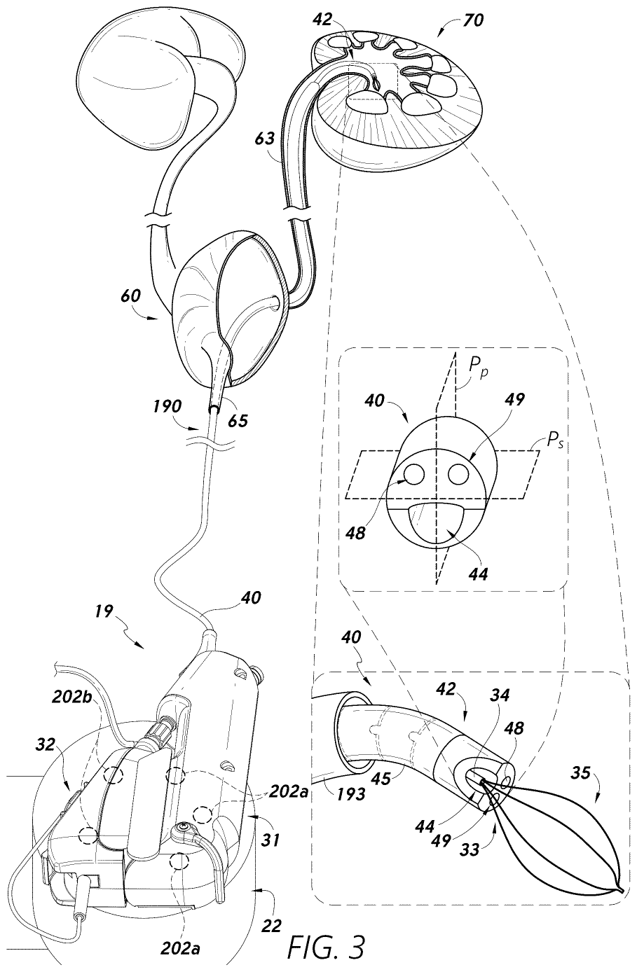

In some examples, the working channel tool assembly 32 may be configured to drive/control various interchangeable tools. An example tool is a basket tool 33 (e.g., a basketing assembly) which may comprise a basket 35 formed of one or more wire tines 36. For example, the basket tool 33 may comprise four wire tines 36 disposed within a basket sheath 34 over a length thereof, wherein the wire tines 36 project from a distal end of the basket sheath 34 to form the basket 35. The wire tines 36 further extend from the proximal end of the basket sheath 34, as shown in FIG. 3. The wire tines 36 and the basket sheath 34 can be coupled to respective drive inputs (not shown) of the working channel tool assembly 32. The wire tines 36 may be configured to be slidable within the basket sheath 34, subject to some amount of frictional resistance, and be biased toward expansion of the basket 35 at a distal end of the basket sheath 34. When the wire tines 36 are extended out of the basket sheath 34 at the distal end (i.e., more of the wire tines 36 are pushed out of the basket sheath 34), the basket 35 can open. Conversely, when the wire tines 36 are retracted into the basket sheath 34 at the distal end (i.e., more of the wire tines 36 are pulled back into the basket sheath 34), the basket 35 can close.

Another example tool is a laser tool 37 which may be introduced into the working channel 44, driven to the tissue site, and perform lithotripsy. The laser tool 37 may comprise an optical fiber 38 to guide the laser and a pulse generator 39 for energy transfer configured to fragment a kidney stone in ureteroscopy. Kidney stone fragments may be removed by the basket tool 33 which can be introduced into the working channel 44 after interchangeably pulling out the laser tool 37.

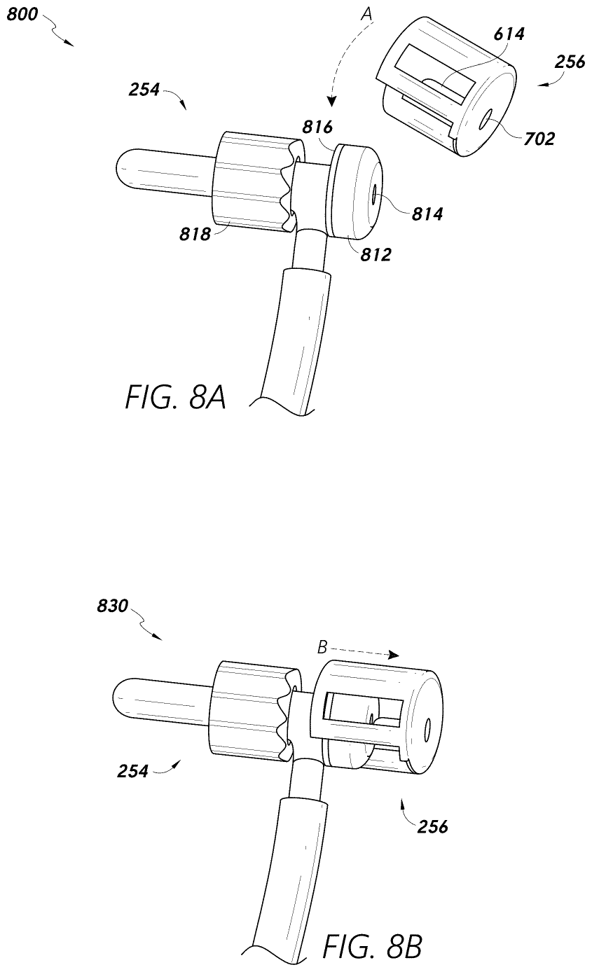

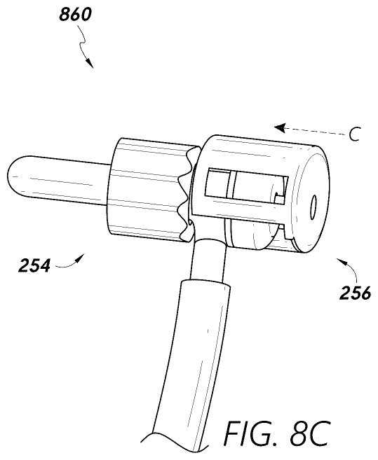

In some examples, the working channel tool assembly 32 may include a valve 254 (e.g., a hemostatic valve or a hemostasis valve) which may be releasably coupled to a proximal end of the working channel 44 to provide a seal around the working channel 44. The seal of the valve 254 can prevent unwanted transit of fluids (e.g., blood, saline solution, irrigation/aspiration fluid, or the like) from flowing out of the working channel 44 and/or prevent outside contaminants from entering the working channel 44, while allowing working channel tools (e.g., the basket tool 33, the laser tool 37 or the like) to pass through the seal. An example valve 254 is shown with respect to FIG. 8A-8C.

To facilitate insertion of a working channel tool into the valve 254 and the working channel 44, an introducer 256 may be coupled to the valve 254. When coupled to the valve 254, the introducer 256 can temporarily open the seal such that the working channel tool may be introduced into the working channel 44. After introduction of the working channel tool into the working channel 44, the introducer 256 may be decoupled from the valve 254 such that the seal can close again.

Traditionally, an introducer 256 is supplied as a separate and removable component to be coupled to the valve 254. As a separate and removable component, the introducer 256 may be lost, misplaced, or become contaminated when dropped and complicate performance of medical procedures. In contrast, as indicated by a boxed introducer assembly 252 containing both the valve 254 and the introducer 256, the working channel tool assembly 32 of the present disclosure contemplates supplying the valve 254 and the introducer 256 as a single introducer assembly 252 with a permanent or a semi-permanent coupling between the valve 254 and the introducer 256. As a single introducer assembly 252, the valve 254 can always have its own introducer 256 that will not be lost or dropped. An example introducer assembly 252 can be seen in a blocked configuration 830 of FIG. 8B and an opened configuration 860 of FIG. 8C.

The scope assembly 519 can be powered through a power interface and/or controlled through a control interface, each or both of which may interface with a robotic arm/component of the robotic system 10. For example, with reference to FIG. 1, an instrument feeder 11 can be attached to the distal end effector 22 of one of the arms 12b to facilitate robotic control/advancement of the scope 40. The scope assembly 519 can be attached to the distal end effector 22 of another one of the arms 12a to facilitate control/advancement of the instrument base 31 and the working channel tool assembly 32.

The scope assembly 519 may further comprise one or more sensors, such as pressure and/or other force-reading sensors, which may be configured to generate signals indicating forces experienced at/by the scope assembly 519.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces 54, 214 of the systems of FIG. 2A can be configured to communicate with one or more device/sensors/systems, such as over a wireless and/or wired network connection. In some examples, the various communication interfaces 54, 214 can implement a wireless technology such as Bluetooth, Wi-Fi, near-field communication (NFC), or the like. Furthermore, in some examples, the various components of the medical system 100 can be connected for data communication, fluid exchange, power exchange, and so on via one or more support cables, tubes, or the like.

### Navigation of Working Channel Tools to Target Site

FIG. 3 illustrates an example ureteroscope (e.g., the scope assembly 519 of FIG. 2B) disposed in portions of the urinary system of a subject, in accordance with some implementations. The scope assembly 519 may comprise a tubular and flexible medical shaft/instrument as a scope 40 configured to be inserted into the anatomy of a subject to capture images of the anatomy and to perform certain tasks using one or more working channels 44 to deploy a variety of working channel tools (e.g., the basket tool 33, the laser tool 37 of FIG. 2B, etc.).

The scope 40 can be advanced to a target site (e.g., a target location, a treatment site, etc.) through an access sheath 193, which may be advanced through the urethra 65, the bladder 60, and the urinary tract 63 to a kidney 70. The distal end of the access sheath 193 may be parked at a position in the urinary tract 63. The access sheath 193 may be placed as far into the renal anatomy as possible, as permitted by the urinary tract 63 path, which may be somewhat tortuous in certain portions thereof.

The scope 40 can be articulable, such as with respect to at least a distal portion 42 of the scope 40, so that the scope 40 can be steered within the human anatomy. In some examples, the scope 40 can be articulated in six degrees of freedom, including XYZ coordinate movement, as well as pitch, yaw, and roll. Certain position sensor(s) (e.g., electromagnetic sensors) of the scope 40, where implemented, may likewise have similar degrees of freedom with respect to the positional information they generate/provide.

For robotic implementations, robotic arms (e.g., the robotic arm(s) 12) of a robotic system (e.g., the robotic system 10) can be configured/configurable to manipulate the scope 40. For example, an end effector of a robotic arm can be coupled to proximal ends of elongate movement members extending along the length of the scope 40 and manipulate the scope 40 using the elongate movement members. The elongate movement members may include one or more pull wires 45 (e.g., pull or push wires), cables, fibers, and/or flexible shafts. For example, the robotic end effector may be configured to actuate multiple pull wires (not shown) coupled to the scope 40 to deflect the distal portion 42 (tip) of the scope 40. The scope 40 can be deflectable in one or two directions within a first/primary plane Pp. The scope 40 can also be deflectable in one or two directions in a second/secondary plane Ps, which may be orthogonal to the primary plane Pp.

Once the scope 40 has navigated to the target site, a working channel tool can be advanced toward the target site using the working channel as a guide. The working channel tool can be introduced into the working channel at any time during the medical procedure, preoperatively or intraoperatively. In some examples, the working channel tool can be interchangeable such that another working channel tool can be advanced to the target site after removal of the working channel tool. For example, a laser tool as a working channel tool may perform lithotripsy in the kidney 70, may be removed from the working channel, and a basket tool as another working channel tool may be introduced into the working channel to remove the fragmented kidney stones from the kidney 70.

The instrument base 31 can generally include an attachment interface having one or more mechanical inputs (e.g., receptacles, pulleys, spools, female inputs, etc.) that are designed to be reciprocally mated with one or more torque couplers on an attachment surface of an end effector 22. In some examples, the instrument base 31 can be configured to attach, mount, or otherwise be connected or coupled to the robotic end effector 22 and one or more drive outputs thereof. The instrument base 31 can include one or more drive inputs configured to engage with and be actuated by corresponding drive outputs to manipulate the scope 40. The elongate movement members can be coupled to the drive inputs and the drive inputs can be configured to control or apply tension to the elongate movement members in response to actuation of the drive outputs.

Previously in relation to FIG. 2A, it was described that a working channel tool assembly 32 can couple to the instrument base 31 and be operated as a single instrument handle. Further, it was described that the instrument handle can be mounted on and operated by an end effector 22 of a single robotic arm (e.g., the robotic arm 12a in FIG. 1). Like the instrument base 31, the working channel tool assembly 32 can be configured to attach, mount, or otherwise be connected or coupled to the robotic end effector 22 and its drive outputs thereof. Like the instrument base 31, the working channel tool assembly 32 can include one or more drive input(s) to manipulate a working channel tool.

The coupling and the mounting of the instrument base 31 and the working channel tool assembly 32 as a single instrument handle can advantageously help align all of the instrument unit and maximally utilize drive outputs 202 of the end effector 22. For example, the example end effector 22 shown has five drive outputs 202, where a first set of drive outputs 202a are engaged with drive inputs of the instrument base 31 and a second set of drive outputs 202b are engaged with drive inputs of the working channel tool assembly 32. Each of the first set of drive outputs 202a can be configured to control one of first deflections on Pp, the second deflections on Ps, and shaft roll. In FIG. 3, the working channel tool assembly 32 is configured with a basket tool (e.g., the basket tool 33 of FIG. 2B). Each of the second set of drive outputs 202b can be configured to control one of insertion/retraction and open/close of the basket tool 33.

### Working Channel Tool Assembly

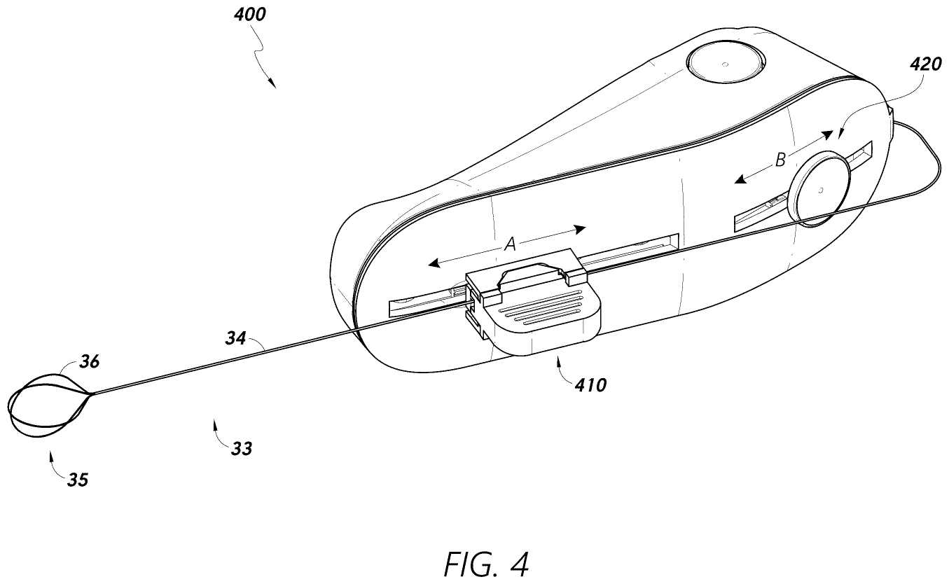

FIG. 4 shows a view 400 of an example working channel tool assembly (e.g., the working channel tool assembly 32 of FIG. 2B), in accordance with some implementations. The working channel tool assembly may include one or more interfaces to facilitate manual control of working channel tool functions. For example, the working channel tool assembly shown has a first interface 410 slidable along a first axis ‘A’ and a second interface 420 slidable along a second axis ‘B’, although other interfaces with other mechanisms are possible.

Manual sliding of the first interface 410 “forward” (i.e., toward the basket 35 as shown) can advance a working channel tool fixed to the first interface 410 toward a target site. Conversely, sliding “backward” (i.e., away from the basket 35 as shown) can retract the fixed working channel tool away from the target site. Similarly, manual sliding of the second interface 420 can advance or retract another working channel tool fixed to the second interface 420 toward or away from the target site. For example, a laser driver fixedly attached to the first interface 410 may advance into a target site when the first interface 410 slides forward and retracts from the target site when the first interface 410 slides backward.

In some examples, the interfaces 410, 420 can be coupled to different parts of the same working channel tool to effectuate relative motion of the parts to each other. FIG. 4 shows a basket tool 33 comprising wire tines 36 telescopically slidable within a basket sheath 34 and forming a basket 35. Here, the first interface 410 can be fixedly coupled with the basket sheath 34 and the second interface 420 can be fixedly coupled with the proximal ends of the wire tines 36. The wire tines 36 are configured to be slidable within the basket sheath 34, subject to some amount of frictional resistance. When the first interface 410 fixed to the basket sheath 34 slides, due to the frictional resistance, it may advance both the basket sheath 34 and the wire tines 36. In contrast, when the second interface 420 fixed to the wire tines 36 slides, the wire tines 36 may overcome the frictional resistance and relatively advance or retract in relation to the stationary basket sheath 34.

As previously described, the wire tines 36 may be biased toward expansion of the basket 35 at a distal end of the basket sheath 34. When the wire tines 36 are pushed out of the basket sheath 34 with forward sliding of the second interface 420, the wire tines 36 can expand and cause the basket 35 to open. Conversely, when the wire tines 36 are retracted into the basket sheath 34 with backward sliding of the second interface 420, the wire tines 36 can contract and cause the basket 35 to close.

### Instrument Handle and Introducer

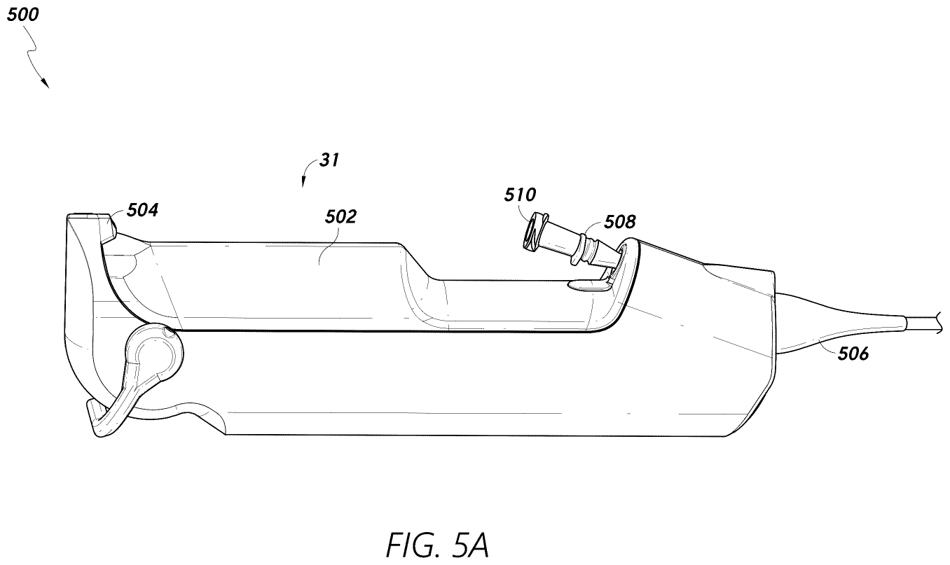

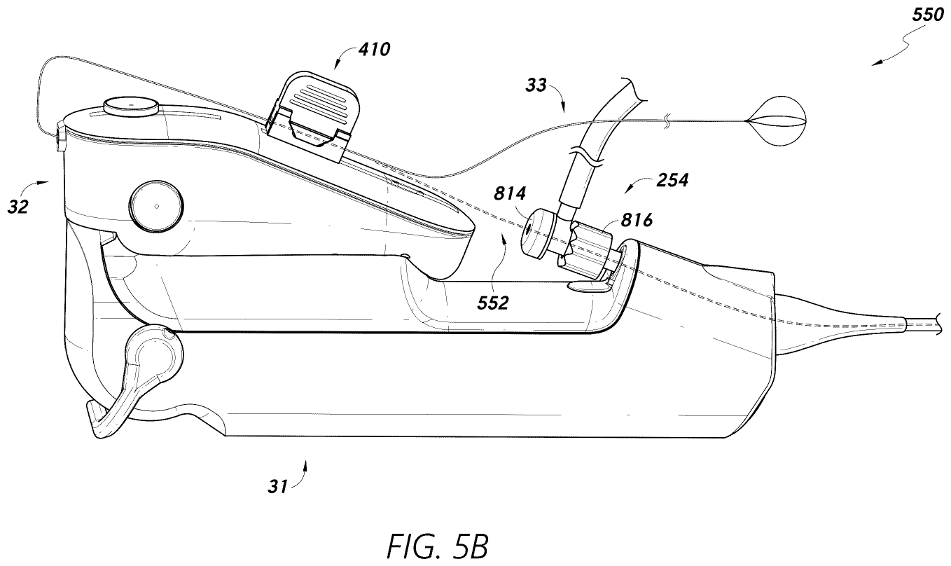

FIG. 5A-5B illustrate an unassembled configuration 500 and an assembled configuration 550, respectively, of an example instrument handle, in accordance with some implementations. The unassembled configuration 500 of FIG. 5A illustrates the instrument base 31 having a mounting surface 502 and a mounting interface 504 (e.g., a locking lip) as coupling mechanisms for mounting a working channel tool assembly 32 (shown in FIG. 5B). Various other coupling mechanisms are possible.

As illustrated, the unassembled configuration 500 shows an endoscope outlet 506 and a working channel tool inlet 510. A scope 40 of FIG. 2B including the working channel 44 may distally extend from the endoscope outlet 506. The working channel tool inlet 510 can be an access point for introduction of the working channel tool and, in some examples, include a locking assembly 508 (e.g., a Luer lock assembly as shown) for coupling with another component.

In FIG. 5B, the assembled configuration 550 has mounted thereon the working channel tool assembly 32 and a valve 254, which may be a hemostatic valve. The working channel tool assembly 32 can be mounted on the instrument base 31 using a counterpart (e.g., a hook to lock onto a lip) to the mounting interface 504 and the valve 254 can be mounted on the instrument base 31 with a counterpart (e.g., a mating Luer lock assembly 818 shown in FIG. 8A, for example) to the locking assembly 508 of FIG. 5A. As shown, the valve 254 can be mounted on the working channel tool inlet 510 (FIG. 5A) such that its seal 814 (e.g., a hemostatic seal) can be positioned on a virtual rail 552 formed between the first interface 410 and the working channel tool inlet 510.

It is noted that the assembled configuration 550 is without an introducer. Generally, the seal 814 may be made of a flexible, compressible material (e.g., such as silicone or rubber) which is effective in sealing function but also resists or otherwise complicates introduction of a working channel tool, such as the basket tool 33 shown, into the seal 814 via an opening defined by the seal. An introducer of the present disclosure (e.g., the introducer 256 of FIG. 2B) that can facilitate introduction of the working channel tool into the opening of the seal 814 will be described in greater detail below.

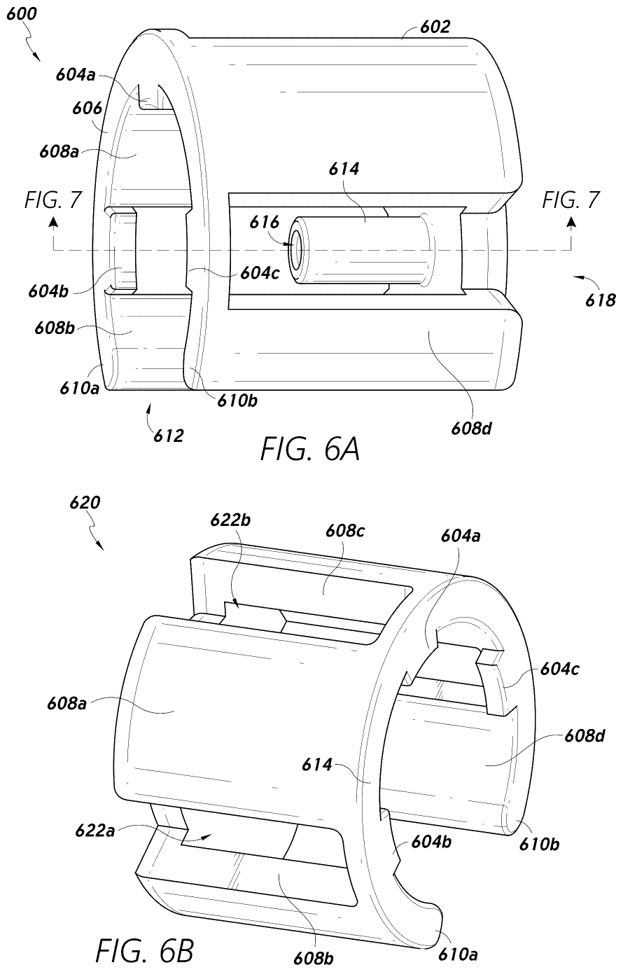

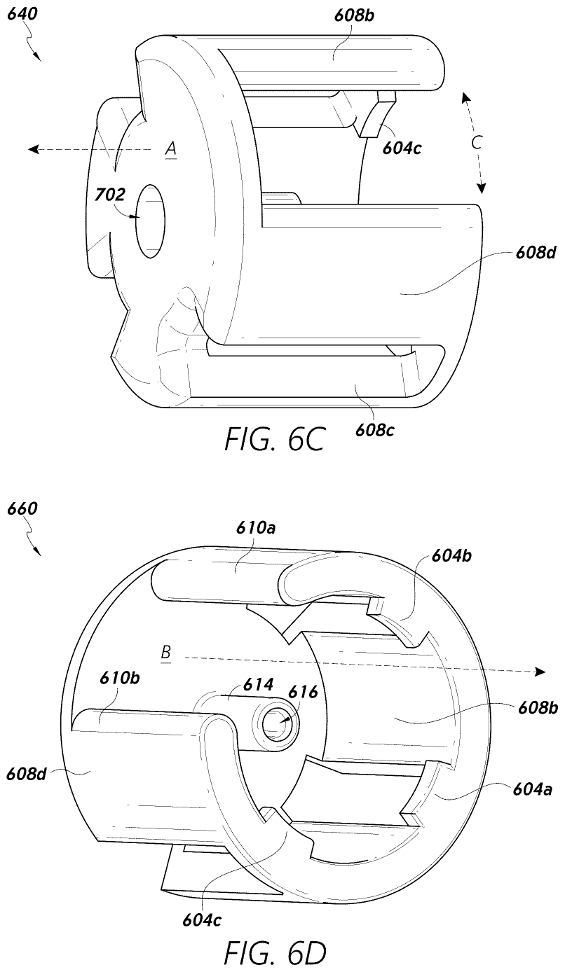

FIG. 6A-6D illustrate various views 600, 620, 640, 660 of an example introducer, in accordance with some implementations. The introducer can be the introducer 256 of FIG. 2B. The introducer can have thereon various structural and functional features each of which will be described in relation to the views 600, 620, 640, 660.

FIG. 6A-6B illustrate a first view 600 and a second view 620 depicting a body 602 having thereon a number of latches (also referred as “lips” or “hooks”) 604 with an introducer rim 606, walls 608 extending from the introducer rim 606, arms 610a, 610b defining a coupling opening 612, a column 614 with a tubular opening (a channel) 616 on the interior of the body 602, and slots 622 formed between the walls 608 (e.g., between angularly adjacent pairs of the walls 608). The walls 608 generally surround the column 614 and, in at least one embodiment, the walls 608 form a C-shaped or crescent geometry. The column 614 and its tubular opening 616 depicted as having circular shapes are exemplary and any oblong or elongated structure with an opening therethrough is possible.

Regarding the latches 604, the introducer shows three latches 604a, 604b, 604c, but any number of latches can be possible. The internally directed latches 604 are examples of a securing mechanism (e.g., a holding mechanism, a hooking mechanism, a latching mechanism, a grabbing mechanism, etc.) that can interface with a valve rim 816 as shown in FIG. 8A to (i) prevent the introducer from falling off the valve 254 and/or (ii) limit a range of sliding-out movement on the valve 254.

The introducer rim 606 can have thereon walls 608 that defines an internal space substantially matching the circumference of the valve rim 816 (FIG. 8A). For example, FIG. 8A shows a valve 254 with a valve head 812 that defines a valve rim 816 with a circumference that substantially matches an internal space defined by the walls 608 of the introducer 256.

As shown in FIG. 6A, the introducer rim 606 can terminate with arms 610a, 610b on respective walls 608b, 608d, where the arms 610a, 610b may be parallel to the column 614. While a cylindrical column 614 (e.g., the column 614 is a cylinder) is illustrated with a circular tubular opening 616, they are exemplary and not to be considered limiting. For example, the column 614 can have a shape of prisms (e.g., trapezoidal, triangular, rectangular, pentagonal, etc.), cones, pillars, pyramids, or any other shapes and the opening 616 can be a conduit, pathway, channel, pipe, tube, duct, passage, lane, etc. having any shape configured to allow introduction of a medical tool therethrough.

The arms 610a, 610b can form the coupling opening 612 that can advantageously allow snapping the introducer 256 onto the head 812 (FIG. 8A) of the valve 254 (FIG. 8A). For example, referring again to FIG. 8A, the introducer 256 can be pressed or rotated onto the valve head 812 along a direction denoted ‘A’ such that the arms 610a, 610b can be forced apart to allow the coupling opening 612 to reach over the valve head 812 and wrap around the valve head 812. In this way, the introducer 256 can become slidably coupled to the valve 254 as shown with introducer assemblies in FIGS. 8B-8C. To allow the coupling, any part or the whole of the body 602 (e.g., the introducer rim 606, walls 608, arms 610a, 610b, or the like) can be made of an elastic material that has some give. In some examples, the elastic material may be a plastic but other materials are also contemplated. Once coupled, the arms 610a, 610b can extend substantially about the valve head 812 and the latches 604 can hook onto the rim 816 to provide slidable coupling between the valve 254 and the introducer 256 while operating as a single introducer assembly 252.

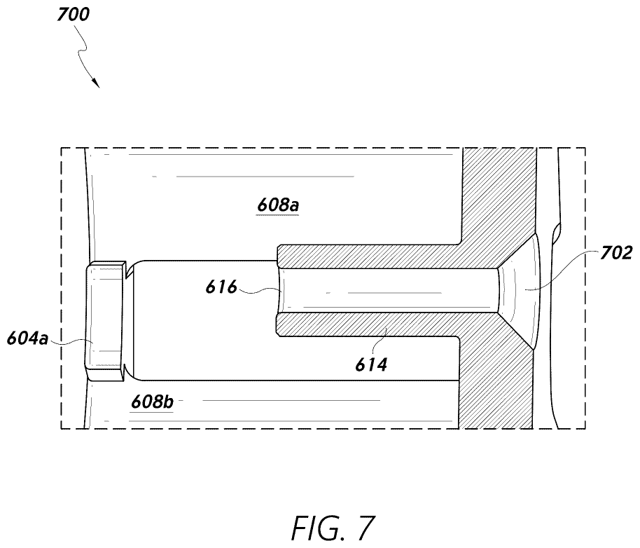

FIG. 6A indicates that a cross-sectional view 700 of the introducer is presented in FIG. 7. The cross-sectional view 700 depicts the column 614 and its tubular opening 616 extending through the column 614. The tubular opening 616 may have a ramped opening 702 on a side of the walls 608a, 608b opposite the side of the latch 604a. Advantageously, the ramped opening 702 can help guide introduction of a working channel tool into the tubular opening 616 with its ramped opening 702.

Referring back to FIG. 6B, the second view 620 shows the slots 622 formed between the walls 608. For example, a first slot 622a is formed between a first wall 608a and a second wall 608b and a second slot 622b is formed between the first wall 608a and a third wall 608c. In some embodiments, as illustrated, the slots 622 can extend to the end of each arm 610a, 610b. In other embodiments, however, one or more of the slots 622 may alternatively comprise an aperture or opening defined between the angularly adjacent walls 608a, 608b, 608c. In such embodiments, the slots 622 may extend from the surface ‘B’ (FIG. 6D), but not to the end of the arms 610a, 610b. The slots 622 can advantageously increase flexibility of the arms 610a, 610b by reducing the amount of elastic material that would resist the pushing apart of the arms 610a, 610b during coupling between the valve 254 and the introducer 256, as shown in FIG. 8A. Furthermore, the slots 622 can facilitate visual inspection of the integrity of the introducer assembly as well as reduce material cost.

FIGS. 6C-6D illustrate a third view 640 and a fourth view 660 showing different perspective views of the example introducer of FIG. 6A-6B. In particular, the third view 640 and the fourth view 660 focus on showing opposing sides or surfaces (denoted ‘A’ and ‘B’) of the introducer. As illustrated, the column 614 and the walls 608a-d each extend from the second surface B. In molding Design for Manufacturing (DFM) where a manufacturing process for the introducer is to be simplified with production cost reduction and potential defect minimization, a mold can be split by the ‘A’ side and the ‘B’ side and pulled in the directions noted by arrows after molding the introducer. In other examples, the introducer is made of a single molded plastic, thereby making it simple and cost effective.

FIG. 6C additionally shows positions denoted ‘C’ on the introducer where gates, which serve as entry points for molten plastic to flow into a molding cavity, can be positioned. Strategic gate placement can help ensure proper flow of the molten plastic thereby avoiding air traps and weld lines. For the introducer, gates can be strategically placed at the positions denoted ‘C’ since little to no operator grabbing action is expected on the arms 610a, 610b during the sliding actions shown in FIGS. 8B-8C. Other DFM considerations, including draft angles selection, ejection mechanisms, cooling channels, or the like may be incorporated into manufacturing of the introducer.

### Operating Introducer Assembly

FIG. 8A-8C illustrate various configurations 800, 830, 860 of an example introducer assembly including a valve and an introducer, in accordance with some implementations. A first configuration 800 illustrates the valve 254 and the introducer 256 separated from each other. As previously described in relation to FIG. 6A, the introducer 256 may be pressed onto, slid onto, rotated onto, or otherwise coupled to the valve 254 such that arms of the introducer 256 can hug (extend at least partially about) the valve head 812, and latches of the introducer 256 can reach over the valve rim 816. The coupling can align the column 614 of the introducer 256 with a seal 814 of the valve 254 and, more particularly, with an opening defined in the seal 814. Additionally, the coupling or the assembly can conFIG. the introducer 256 with limited slidable movement on the valve 254 such that various introducer assembly configurations are possible. In particular, FIG. 8B illustrates a blocked configuration 830 and FIG. 8C illustrates an opened configuration 860, in accordance with some implementations.

In the blocked configuration 830, the introducer 256 is slid backward (slid away from the valve 254) in the direction denoted ‘B’ such that the column 614 of the introducer 256 is pulled away from the seal 814, thereby allowing the opening to the seal 814 to close. The closed seal 814 can complicate introduction of working channel tools by blocking the working channel tools inserted into an inlet (e.g., the ramped opening 702 of FIG. 7) to the column 614 at the closed seal 814.

In the opened configuration 860, the introducer 256 is slid forward (slid toward the valve 254) in the direction denoted ‘C’ such that the column 614 of the introducer 256 is pushed into (penetrates) the seal 814 and thereby opens the seal 814. The opened seal 814 can facilitate introduction of the working channel tools into the inlet by permitting access into the valve 254 though the column 614.

In some examples, the introducer 256 can be permanently or semi-permanently coupled to the valve 254 after the coupling, as the introducer assembly. Advantageously, the introducer 256 is user friendly, easy to manufacture, and easy to operate (e.g., does not involve rotation or other mechanisms).

### Introducer Assembly Process

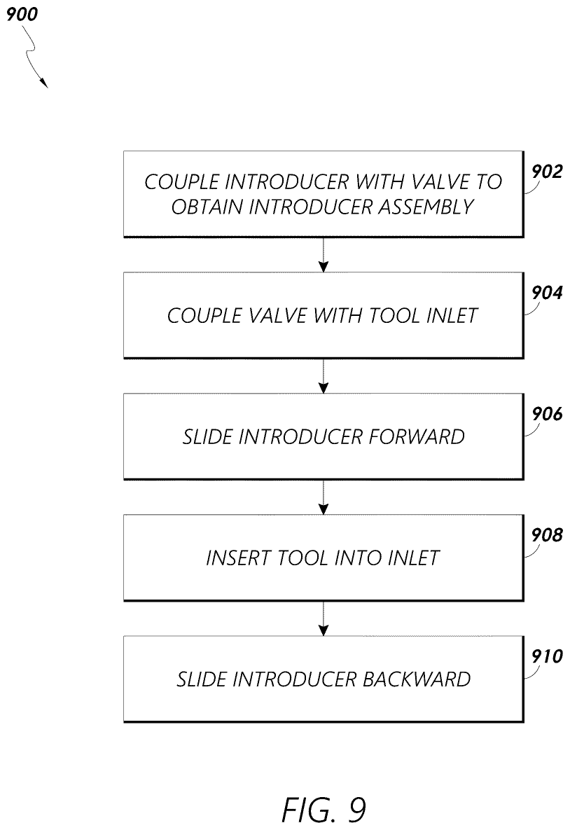

FIG. 9 illustrates a flow diagram for an example process 900 for assembling and operating the example introducer assembly of FIG. 8A-8C, in accordance with some implementations. The process 900 may include additional blocks or fewer blocks and, in some examples, blocks out of order compared to the order shown.

At block 902, the process 900 involves coupling an introducer with a valve to obtain an introducer assembly. The introducer, the valve, and the introducer assembly can be, respectively, the introducer 256, the valve 254, and the introducer assembly 252 of FIG. 2B. In some examples, a manufacturer of the valve or the introducer may manufacture and supply the introducer assembly as a whole component.

At block 904, the process 900 involves coupling the valve with a working channel tool inlet (e.g., the working channel tool inlet 510 of FIG. 5A). The valve may be part of the introducer assembly of block 902 which has the introducer already coupled thereon. In some examples, the valve may be first coupled to the working channel tool inlet and the introducer may be coupled on the valve thereafter. As described in relation to FIG. 5B, the valve may be coupled with the working channel tool inlet using one or more locking assemblies, such as Luer locks.

At block 906, the process 900 involves sliding the introducer of the introducer assembly forward (e.g., in the direction ‘C’ shown in FIG. 8C). The sliding forward of the introducer can cause a seal of the valve to open, thereby facilitating introduction of a working channel tool into the valve.

At block 908, the process 900 involves inserting/introducing a working channel tool into the working channel tool inlet. The working channel tool can be a basket tool, a laser tool, or any other flexible/bendable shafts/shaft-type tools insertable into the working channel. In some examples, the shafts/shaft-type tools may have a sensor (e.g., an ultrasound sensor, an imaging sensor, etc.) positioned at a distal end of the shaft-type tool.

At block 910, the process 900 involves sliding the introducer of the introducer assembly backward (e.g., in the direction ‘B’ shown in FIG. 8B). The sliding backward of the introducer can cause the seal of the valve to close, thereby providing hemostasis.

The introducer thus far described may be referred as a “slide-in design” introducer. The remaining figures illustrate and the following descriptions provide introducers having different designs based on different coupling mechanisms compared to the slide-in design. It will be understood that the term “slide-in design” is used to refer to the above described design but is not to be construed as excluding the other designs from having a slidable aspect, for example, to open a seal of a valve. All of following introducer designs will be described with respect to the assembled configuration 550 of FIG. 5B where an introducer can facilitate introduction of a working channel tool into the seal 814 of a valve 254.

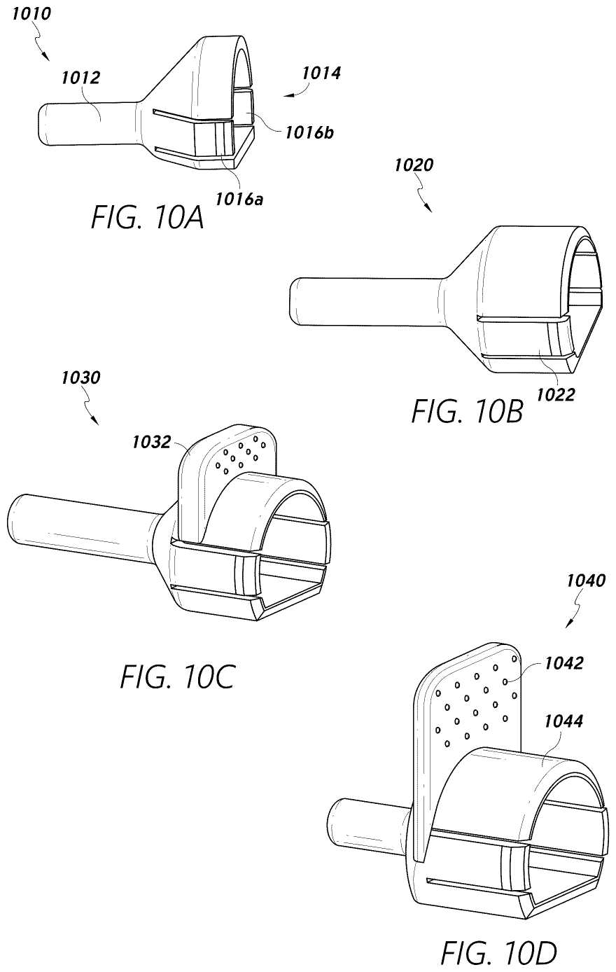

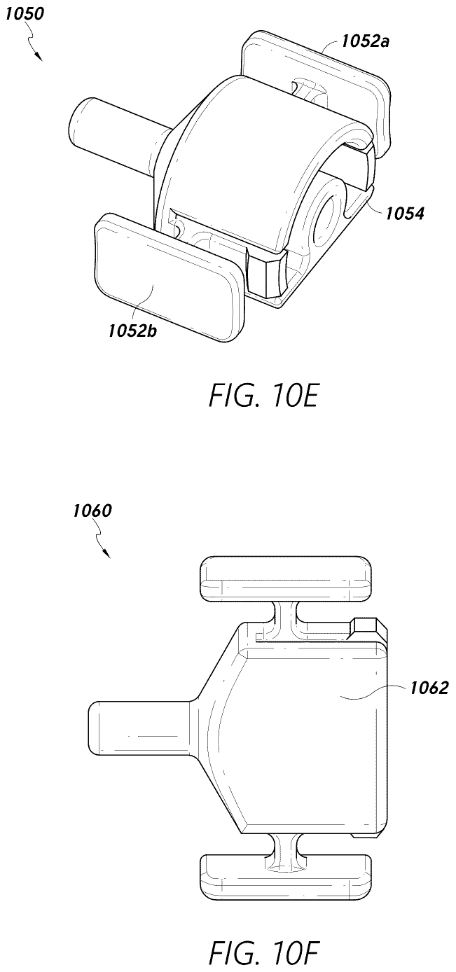

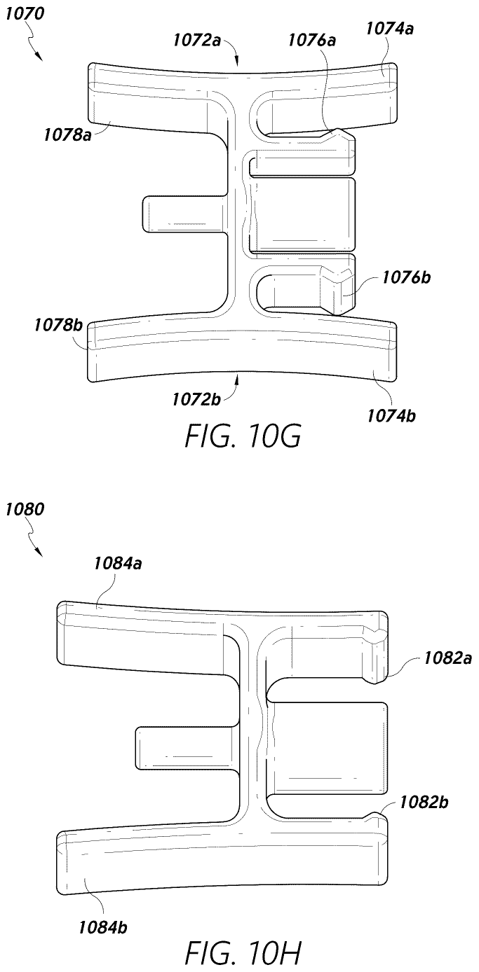

FIG. 10A-10H illustrate example introducers having various latching designs, in accordance with some implementations. In FIG. 10A, a first latch introducer 1010 has a column 1012 which can be inserted into the seal 814 to open the seal 814 for insertion of a working channel tool. The first latch introducer 1010 further defines an opening 1014 which may be ramped to help guide introduction of the working channel tool.

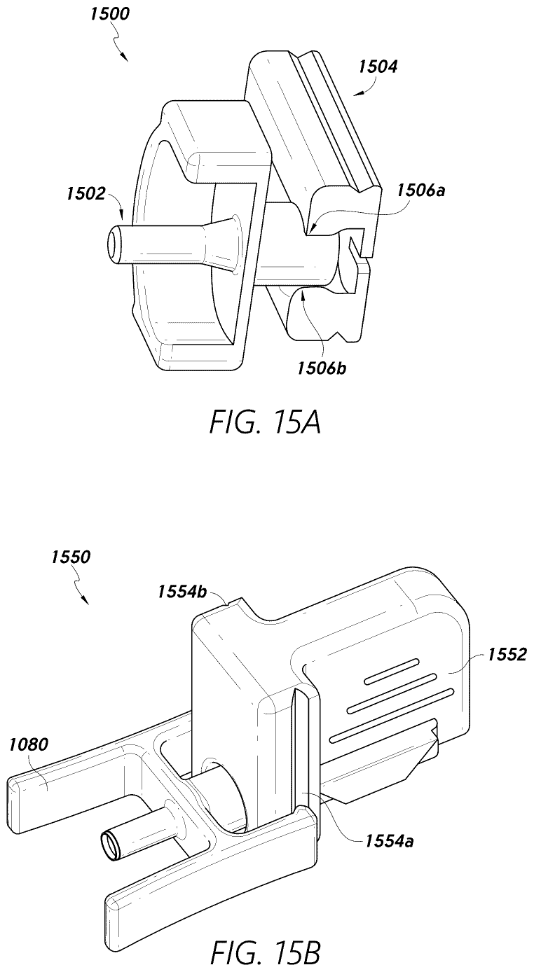

Additionally, the first latch introducer 1010 has one or more snap features 1016a, 1016b that would latch/lock to a recess feature or a notch formed on a hub (e.g., a first example hub 1500 of FIG. 15A) such that the first latch introducer 1010 is not easily separated and misplaced/lost. The snap features 1016a, 1016b can be made of elastic material such that pressing the first latch introducer 1010 into the hub can deform the snap features 1016a, 1016b temporarily to push the snap features 1016a, 1016 against the recess feature or the notch, which can hug the first latch introducer 1010 in place.

In FIG. 10B, a second latch introducer 1020, when compared to the first latch introducer 1010, provides more/longer grip room 1022 for its snap features, thereby making it easier to actuate the snap features.

In FIG. 10C, a third latch introducer 1030, when compared to the second latch introducer 1020, additionally provides a tab 1032 to improve handling.

In FIG. 10D, a fourth latch introducer 1040, when compared to the third latch introducer 1030, provides an enlarged tab 1042 and/or an enlarged back section 1044 to further improve handling.

In FIG. 10E, a fifth latch introducer 1050, when compared to the fourth latch introducer 1040, provides side tabs 1052a, 1052b to further improve handling.

In FIG. 10F, a sixth latch introducer 1060, when compared to the fifth latch introducer 1050, shows its body 1062 made flat for symmetry such that the sixth latch introducer 1060 may be used with multiple orientations.

In FIG. 10G, a seventh latch introducer 1070, when compared to the sixth latch introducer 1060, provides enlarged gripping surfaces 1072a, 1072b. The gripping surfaces 1072a, 1072 have thereon actuatable (pinchable, pressable) features 1074a, 1074b or spreadable/expandable features 1078a, 1078b that would cause, respectively, bending or deformation of corresponding internal latches 1076a, 1076b as snap features (e.g., the snap features 1016a, 1016b of the first latch introducer 1010 in FIG. 10A). When left unpinched, the pinchable features 1074a, 1074b would leave the corresponding internal latches 1076a, 1076b to latch onto notches of a hub (e.g., a second example hub 1550 of FIG. 15B showing a latched configuration). However, when pinched, the pinchable features 1074a, 1074b would cause the corresponding internal latches 1076a, 1076b to unlatch from the notches of the hub, thereby making the seventh latch introducer 1070 easier to attach and release to the hub.

In FIG. 10H, an eighth latch introducer 1080 provides external latches 1082a, 1082b instead of the internal latches 1076a, 1076b of the seventh latch introducer 1070. The external latches 1082a, 1082b can have corresponding pinchable features 1084a, 1084b thereon that, when left unpinched, would leave the corresponding external latches 1082a, 1082b to latch onto notches of a hub (e.g., the first example hub 1500 of FIG. 15A showing a latched configuration). However, when pinched (i.e., forced radially inward toward each other), the pinchable features 1084a, 1084b would cause the corresponding external latches 1082a, 1082b to unlatch from the notches of the hub.

While the various latch introducers shown illustrate the latch introducers having the snap features and hubs having corresponding recess features, it will be understood that the latch introducers may have the recess features and the hubs may have corresponding recess features, or other combinations of snap features and recess features.

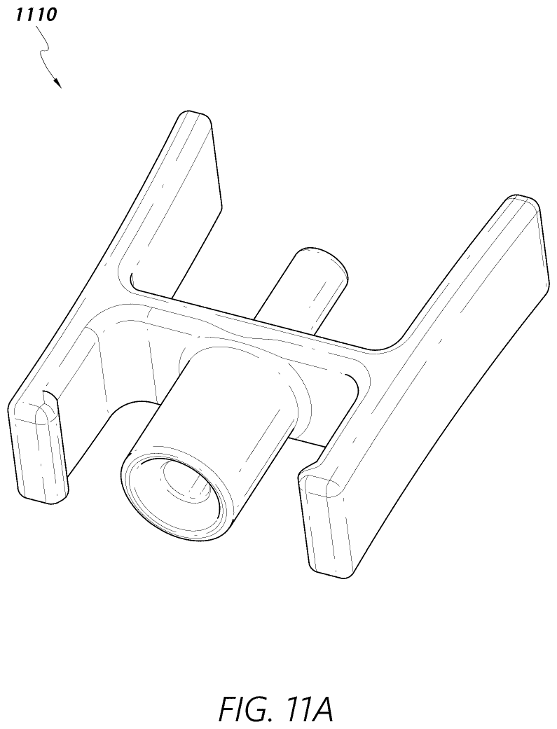

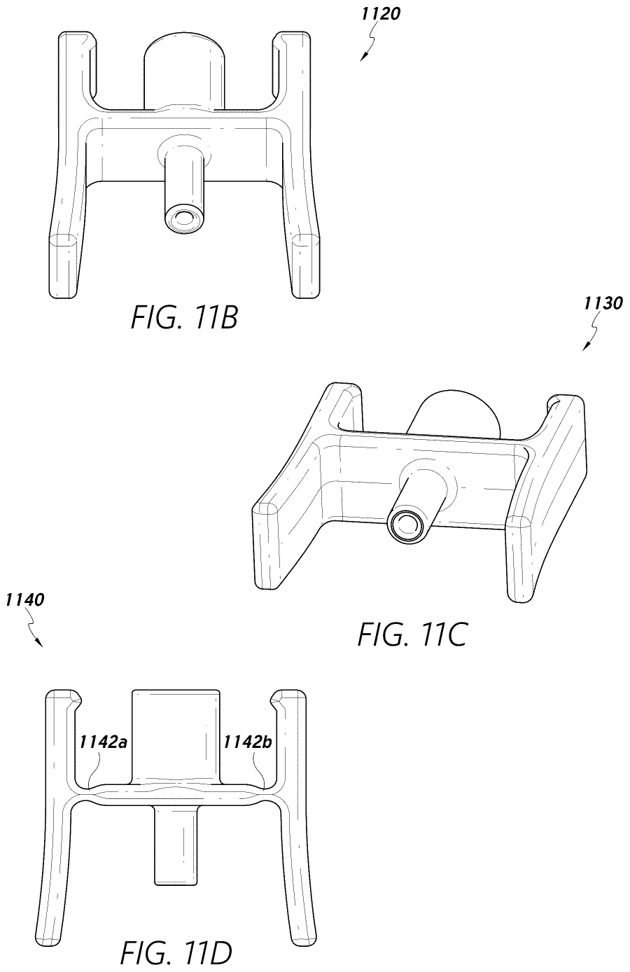

FIG. 11A-11D illustrates various views, respectively 1110, 1120, 1130, 1140, of the latching design of the eighth latch introducer 1080 of FIG. 10H, in accordance with some implementations. FIG. 11D illustrates a top view 1140 which has a modified version of the eighth latch introducer 1080 of FIG. 10H. The modified version shows strategic placement of thinner walls 1142a, 1142b that can operate as living hinges and help facilitate latching and release of the external latches 1082a, 1082b in FIG. 10H through reduction of pinching force on the pinchable features 1084a, 1084b.

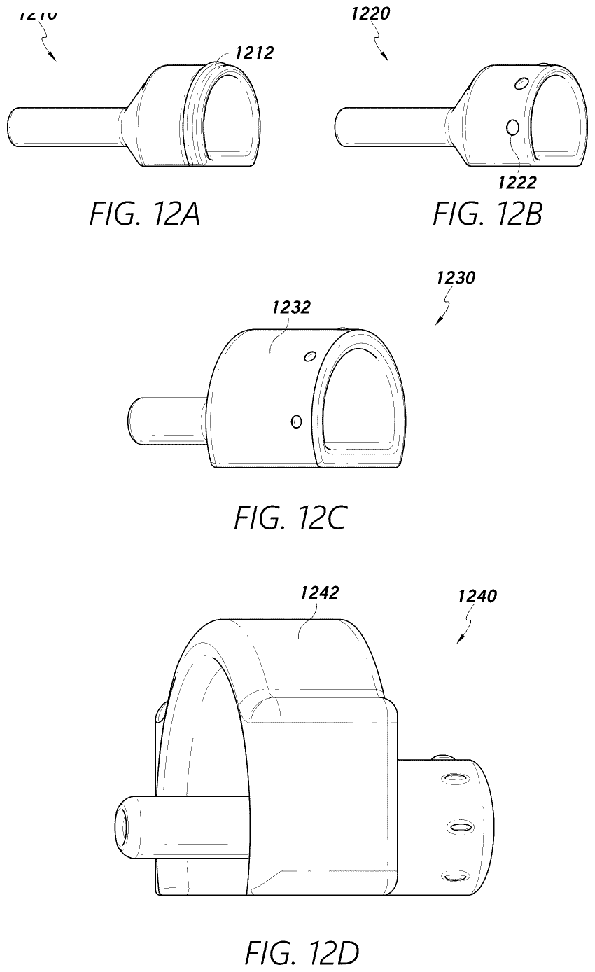

FIG. 12A-12D illustrates example introducers having various press-fit designs, in accordance with some implementations. In FIG. 12A, a first press-fit introducer 1210 has a protrusion feature 1212 that would cause enough friction when pressed into a receiving feature formed on a hub (e.g., a first example hub 1500 of FIG. 15A) such that the first press-fit introducer 1210 is not easily separated and misplaced/lost. The protrusion feature 1212 can be made of elastic material with sufficient give that allows press-fit with sufficient friction such that the hub can hug the first press-fit introducer 1210 in place.

In FIG. 12B, a second press-fit introducer 1220, when compared to the band-like protrusion feature 1212 of the first press-fit introducer 1210, provides droplet-like protrusion features 1222 that make it easier to press the droplet-like protrusion features 1222 into the hub.

In FIG. 12C, a third press-fit introducer 1230, when compared to the second press-fit introducer 1220, provides an enlarged back section 1232 to improve handling.

In FIG. 12D, a fourth press-fit introducer 1240, when compared to the third press-fit introducer 1230, provides an arched surface 1242 to further improve handling.

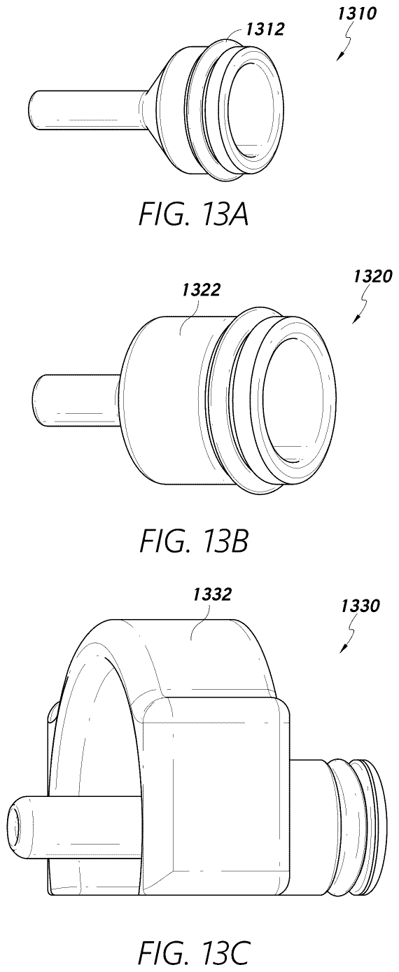

FIG. 13A-13C illustrate example introducers having various O-ring-based designs, in accordance with some implementations. In FIG. 13A, a first O-ring introducer 1310 has an O-ring 1312 that would cause enough friction when pressed into a receiving feature formed on a hub (e.g., a first example hub 1500 of FIG. 15A) such that the first O-ring introducer 1310 is not easily separated and misplaced/lost. The O-ring 1312 is elastic with sufficient give to allow pressing the first O-ring introducer 1310 into the hub with sufficient friction such that the hub can hold the first O-ring introducer 1310 in place via an interference fit.

In FIG. 13B, a second O-ring introducer 1320, when compared to the first O-ring introducer 1310, provides a longer main body 1322 to improve handling.

In FIG. 13C, a third O-ring introducer 1330, when compared to the second O-ring introducer 1320, provides an arched surface 1332 to further improve handling.

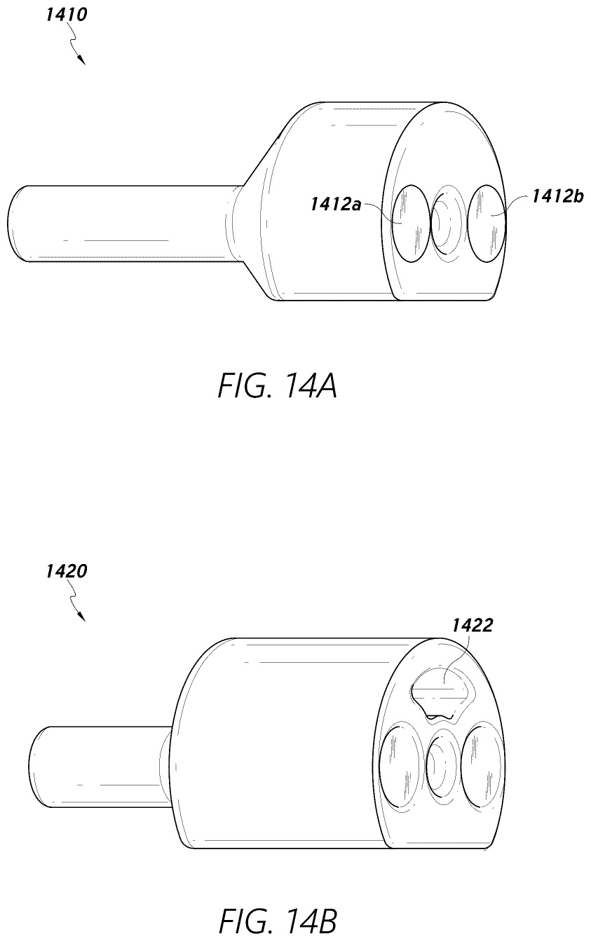

FIG. 14A-14B illustrates example introducers having various magnetic designs, in accordance with some implementations. In FIG. 14A, a first magnetic introducer 1410 has one or more magnets 1412a, 1412b that would magnetically, releasably attach onto a corresponding one or more magnets on a hub such that the first magnetic introducer 1410 is not easily separated and misplaced/lost.

In FIG. 14B, a second magnetic introducer 1420, when compared to the first magnetic introducer 1410, additionally provides an aligning feature 1422 to receive a corresponding feature formed on a hub. The aligning feature 1422 can help orient the second magnetic introducer 1420 in relation to the hub.

FIG. 15A-15B illustrate example hubs to which an introducer can be releasably coupled, in accordance with some implementations. A first example hub 1500 of FIG. 15A illustrates an introducer 1502, which can be any introducer described herein, held by first hub features 1506a, 1506b. The first hub features 1506a, 1506b can be the recess features, notch features, hugging features, magnetic features, or any features described to releasably hold the introducer 1502. The first hub features 1506a, 1506b hug/embrace/squeeze/clasp/hold corresponding features of the introducer 1502.

A second example hub 1550 of FIG. 15B illustrates an introducer (e.g., the eighth latch introducer 1080 of FIG. 10H) held by second hub features 1554a, 1554b. In some examples, the second hub features 1554a, 1554b can be formed on a body 1552 of the first interface 410 of FIG. 5B or, alternatively, on a separate piece that attaches to the first interface 410. Therefore, after introduction of a working channel tool into a seal of a valve, the introducer can be secured on the first interface 410. In comparison to the first example hub 1500 of FIG. 15A, the second hub features 1554a, 1554b of the second example hub 1550 can be hugged/embraced/squeezed/clasped/held by corresponding features (e.g., the external latches 1082a, 1082b), as shown. Many variations of hubs are possible, including any feature combinations of the example hubs.

### Additional Embodiments

Embodiments disclosed herein include:

A. A tool introducer assembly that includes a valve having a valve head that provides a seal, and an introducer that includes a column that defines a channel that extends therethrough, and a securing mechanism configured to attach the introducer to the valve, wherein the valve and the introducer are slidably coupled with a range of motion limited by the securing mechanism, and wherein, when the valve and the introducer are coupled, the column of the introducer is aligned with an opening defined by the seal.

B. A device for introducing a flexible elongate tool into a valve, the device including a column molded on and extending from a surface and defining a channel that extends through the column, a set of walls extending from the surface parallel to and at least partially surrounding the column, the set of walls terminating at and forming a crescent at a side opposite the surface in relation to the column, the crescent providing an opposing surface that is parallel to the surface, and at least one latch formed on the crescent and facing radially inward and toward the column.

C. A method of introducing a working channel tool into a valve, the method including assembling an introducer with the valve to obtain an introducer assembly, the introducer including a column that defines a channel that extends therethrough, and at least one latch that secures the introducer to the valve. The method further including sliding the introducer toward the valve and thereby opening a seal on the valve as the column penetrates an opening defined in the seal, sliding the introducer away from the valve and thereby closing the seal as the column withdraws from the opening, and preventing the introducer from decoupling from the valve with the at least one latch.

Each of embodiments A, B, and C may have one or more of the following additional elements in any combination: Element 1: wherein the securing mechanism includes at least one latch. Element 2: wherein the at least one latch extends radially inward and toward the column, and the at least one latch is engageable with a valve rim defined on the valve head. Element 3: wherein the at least one latch includes two opposing latches. Element 4: wherein the introducer further includes a set of arms that extend parallel to the column and help define a coupling opening for the introducer. Element 5: wherein the introducer is pressed onto the valve at the coupling opening to attach the introducer to the valve and, when coupled, the set of arms extend at least partially about the valve head and are engageable with a valve rim defined by the valve head. Element 6: wherein the set of arms are forced away from each other to enlarge the coupling opening and thereby allow the introducer to be received onto the valve. Element 7: wherein at least a portion of the introducer is made of an elastic material such that the set of arms can revert back to an original state after attaching the introducer to the valve. Element 8: wherein the introducer further includes a plurality of walls connecting the securing mechanism and the column, and a slot defined between each pair of angularly adjacent wall of the plurality of walls. Element 9: wherein, once attached to the valve, the introducer is longitudinally slidably relative to the valve between a blocked configuration, where the column is separated from the seal and the seal is thereby closed, and an opened configuration, where the column is received within the opening and the seal is thereby open. Element 10: wherein the introducer is slidable toward the valve to penetrate and open the seal with the column. Element 11: wherein the introducer is made of molded plastic. Element 12: wherein the channel defines a ramped opening configured to receive a flexible tool.

Element 13: wherein the at least one latch includes at least two latches angularly offset from each other on the crescent. Element 14: wherein the set of walls defines a coupling opening and the device is attachable to a head of the valve by forcing the set of walls over the head at the coupling opening, and thereby at least partially surrounding the head. Element 15: wherein the column is aligned with an opening to a seal of the valve when the device is attached to the head. Element 16: wherein, once attached to the valve, the device is longitudinally slidably relative to the valve between a blocked configuration, where the column is separated from the seal and the seal is thereby closed, and an opened configuration, where the column is received within the opening and the seal is thereby open.

Element 17: wherein sliding the introducer toward the valve and thereby opening the seal further comprises introducing the working channel tool into the seal through the column.

By way of non-limiting example, exemplary combinations applicable to A, B, and C include: Element 1 with Element 2; Element 2 with Element 3; Element 4 with Element 5; Element 4 with Element 6; Element 4 with Element 7; Element 14 with Element 15; and Element 15 with Element 16.

Depending on the embodiment, certain acts, events, or functions of any of the processes or algorithms described herein can be performed in a different sequence, may be added, merged, or left out altogether. In addition, various features of different disclosed embodiments can be combined to form additional embodiments, which are part of this disclosure. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one. ” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A tool introducer assembly, comprising:
a valve having a valve head that provides a seal; and
an introducer that includes:
a column that defines a channel that extends therethrough; and
a securing mechanism configured to attach the introducer to the valve,
wherein the valve and the introducer are slidably coupled with a range of motion limited by the securing mechanism, and
wherein, when the valve and the introducer are coupled, the column of the introducer is aligned with an opening defined by the seal.

2. The tool introducer assembly of claim 1, wherein the securing mechanism includes at least one latch.

3. The tool introducer assembly of claim 2, wherein the at least one latch extends radially inward and toward the column, and the at least one latch is engageable with a valve rim defined on the valve head.

4. The tool introducer assembly of claim 3, wherein the at least one latch includes two opposing latches.

5. The tool introducer assembly of claim 1, wherein the introducer further includes a set of arms that extend parallel to the column and help define a coupling opening for the introducer.

6. The tool introducer assembly of claim 5, wherein the introducer is pressed onto the valve at the coupling opening to attach the introducer to the valve and, when coupled, the set of arms extend at least partially about the valve head and are engageable with a valve rim defined by the valve head.

7. The tool introducer assembly of claim 5, wherein the set of arms are forced away from each other to enlarge the coupling opening and thereby allow the introducer to be received onto the valve.

8. The tool introducer tool introducer assembly of claim 5, wherein at least a portion of the introducer is made of an elastic material such that the set of arms can revert back to an original state after attaching the introducer to the valve.

9. The tool introducer assembly of claim 1, wherein the introducer further includes:
a plurality of walls connecting the securing mechanism and the column; and
a slot defined between each pair of angularly adjacent wall of the plurality of walls.

10. The tool introducer assembly of claim 1, wherein, once attached to the valve, the introducer is longitudinally slidably relative to the valve between a blocked configuration, where the column is separated from the seal and the seal is thereby closed, and an opened configuration, where the column is received within the opening and the seal is thereby open.

11. The tool introducer assembly of claim 1, wherein the introducer is slidable toward the valve to penetrate and open the seal with the column.

12. The tool introducer assembly of claim 1, wherein the introducer is made of molded plastic.

13. The tool introducer assembly of claim 1, wherein the channel defines a ramped opening configured to receive a flexible tool.

14. A device for introducing a flexible elongate tool into a valve, the device comprising:
a column molded on and extending from a surface and defining a channel that extends through the column;
a set of walls extending from the surface parallel to and at least partially surrounding the column, the set of walls terminating at and forming a crescent at a side opposite the surface in relation to the column, the crescent providing an opposing surface that is parallel to the surface; and
at least one latch formed on the crescent and facing radially inward and toward the column.

15. The device of claim 14, wherein the at least one latch includes at least two latches angularly offset from each other on the crescent.

16. The device of claim 14, wherein the set of walls defines a coupling opening and the device is attachable to a head of the valve by forcing the set of walls over the head at the coupling opening, and thereby at least partially surrounding the head.

17. The device of claim 16, wherein the column is aligned with an opening to a seal of the valve when the device is attached to the head.

18. The device of claim 17, wherein, once attached to the valve, the device is longitudinally slidably relative to the valve between a blocked configuration, where the column is separated from the seal and the seal is thereby closed, and an opened configuration, where the column is received within the opening and the seal is thereby open.

19. A method of introducing a working channel tool into a valve, the method comprising:
assembling an introducer with the valve to obtain an introducer assembly, the introducer including:
a column that defines a channel that extends therethrough; and
at least one latch that secures the introducer to the valve;
sliding the introducer toward the valve and thereby opening a seal on the valve as the column penetrates an opening defined in the seal;
sliding the introducer away from the valve and thereby closing the seal as the column withdraws from the opening; and
preventing the introducer from decoupling from the valve with the at least one latch.

20. The method of claim 19, wherein sliding the introducer toward the valve and thereby opening the seal further comprises introducing the working channel tool into the seal through the column.
